(19) United States
(12) Patent Application Publication
(10) Pub. No.: US 2023/0310063 A1
(43) Pub. Date: Oct. 5, 2023

(54) THERMAL CUTTING ELEMENTS, ELECTROSURGICAL INSTRUMENTS INCLUDING THERMAL CUTTING ELEMENTS, AND METHODS OF MANUFACTURING

(71) Applicant: Covidien LP, Mansfield, MA (US)

(72) Inventors: James D. Allen, IV, Broomfield, CO (US); William E. Robinson, Boulder, CO (US); Daniel A. Joseph, Golden, CO (US); John A. Hammerland, Arvada, CO (US); Kenneth E. Netzel, Loveland, CO (US)

(21) Appl. No.: 18/023,799

(22) PCT Filed: Sep. 1, 2021
(86) PCT No.: PCT/US2021/048658

§ 371 Date: Feb. 28, 2023

Related U.S. Application Data
(60) Provisional application No. 63/073,397, filed on Sep. 1, 2020.
(60) Provisional application No. 63/073,412, filed on Sep. 1, 2020.

(51) Int. Cl. A61B 18/14

(52) U.S. Cl. CPC A61B 18/1445; A61B 2018/00083; A61B 2018/1452

(57) ABSTRACT

An end effector assembly for an electrosurgical instrument includes a pair of opposing jaw members each having a jaw housing supporting an electrically conductive tissue engaging surface thereon disposed in opposition relative to one another. The electrically conductive tissue engaging surfaces are adapted to connect to an electrosurgical energy source. A thermal cutting element is disposed the electrically conductive tissue engaging surface and is independently activatable relative to the electrically conductive tissue engaging surfaces and is adapted to connect to the electrosurgical energy source. The thermal cutting element is exposed along the length of the electrically conductive tissue engaging surface and includes an exposed distal end extending through a distal end of the jaw housing. The exposed distal end of the thermal cutting element is configured to facilitate tissue dissection upon activation thereof.

### FIELD

The present disclosure relates to surgical instruments and, more particularly, to thermal cutting elements, electrosurgical instruments including thermal cutting elements, and methods of manufacturing thermal cutting elements.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is an end effector for a surgical instrument that includes a pair of opposing jaw members each having a jaw housing supporting an electrically conductive tissue engaging surface thereon disposed in opposition relative to one another. One (or both) of the pair of jaw members is movable relative to the other of the pair of jaw members to grasp tissue therebetween. The electrically conductive tissue engaging surfaces are adapted to connect to an electrosurgical energy source. A thermal cutting element is disposed in one (or both) of the electrically conductive tissue engaging surfaces and is independently activatable relative to the electrically conductive tissue engaging surfaces and adapted to connect to the electrosurgical energy source. The thermal cutting element is exposed along the length of the electrically conductive tissue engaging surface and includes an exposed distal end extending through a distal end of the jaw housing. The exposed distal end of the thermal cutting element is configured to facilitate tissue dissection upon activation thereof.

In aspects according to the present disclosure, the thermal cutting element is seated within an insulator disposed in the at least one electrically conductive tissue engaging surface. In other aspects according to the present disclosure, the thermal cutting element is raised relative to the insulator. In other aspects according to the present disclosure, the thermal cutting element is a trace directly adhered to the insulator or the seal surface.

In aspects according to the present disclosure, a portion of the thermal cutting element includes a beveled surface. In other aspects according to the present disclosure, the thermal cutting element includes a cutting spine disposed along a length thereof having a pair of opposing beveled edges extending away therefrom that are configured to slough tissue away from the cutting spine once the tissue is cut.

In aspects according to the present disclosure, the exposed distal end of the thermal cutting element includes at least one beveled edge. In other aspects according to the present disclosure, the thermal cutting element extends relative to a distal end of the jaw housing.

Provided in accordance with aspects of the present disclosure is an end effector for a surgical instrument that includes a pair of opposing jaw members each having a jaw housing supporting an electrically conductive tissue engaging surface thereon disposed in opposition relative to one another. One (or both) of the pair of jaw members is movable relative to the other of the pair of jaw members to grasp tissue therebetween. The electrically conductive tissue engaging surfaces are adapted to connect to an electrosurgical energy source. A thermal cutting element is disposed through the electrically conductive tissue engaging surface and has a portion thereof disposed through the jaw housing to an opposite end thereof. The thermal cutting element is independently activatable relative to the electrically conductive tissue engaging surfaces and is adapted to connect to the electrosurgical energy source. The thermal cutting element is exposed along the length of the electrically conductive tissue engaging surface and is exposed through the jaw housing on the opposite end thereof.

In aspects according to the present disclosure, the thermal cutting element includes an exposed distal end extending through a distal end of the jaw housing, the exposed distal end of the thermal cutting element configured to facilitate tissue dissection upon activation thereof. In other aspects according to the present disclosure, the exposed portion of the thermal cutting element extending through the jaw housing on the opposite end thereof is configured to facilitate back-scoring of tissue along an edge thereof.

In aspects according to the present disclosure, the thermal cutting element is secured within an insulator disposed in the electrically conductive tissue engaging surface. In other aspects according to the present disclosure, the thermal cutting element is raised relative to the insulator.

In aspects according to the present disclosure, a portion of the thermal cutting element includes a beveled surface. In other aspects according to the present disclosure, the thermal cutting element includes a cutting spine disposed along a length thereof having a pair of opposing beveled edges extending away therefrom that are configured to slough tissue away from the cutting spine once the tissue is cut.

In other aspects according to the present disclosure, the exposed distal end of the thermal cutting element includes at least one beveled edge. In other aspects according to the present disclosure, the thermal cutting element extends relative to a distal end of the jaw housing.

Provided in accordance with aspects of the present disclosure is an end effector for a surgical instrument that includes a pair of opposing first and second jaw members each including a jaw housing supporting an electrically conductive tissue engaging surface thereon. The electrically conductive tissue engaging surfaces of the first and second jaw members are disposed in opposition relative to one another and the first jaw member is movable relative to the second jaw member to grasp tissue therebetween. The electrically conductive tissue engaging surfaces of the first and second jaw members are adapted to connect to an electrosurgical energy source. A first thermal cutting element is disposed on the electrically conductive tissue engaging surface of the first jaw member and has a portion thereof that partially wraps around a distal end of the jaw housing of the first jaw member.

The thermal cutting element is independently activatable relative to the electrically conductive tissue engaging surfaces and is adapted to connect to the electrosurgical energy source. The thermal cutting element is exposed along the length of the electrically conductive tissue engaging surface of the first jaw member and is exposed at the distal end of the jaw housing of the first jaw member.

In aspects according the present disclosure, the end effector assembly further includes a second thermal cutting element disposed on the electrically conductive tissue engaging surface of the second jaw member. The second thermal cutting element has a portion that partially wraps around a distal end of the jaw housing of the second jaw member. The second thermal cutting element is independently activatable along with the first thermal cutting element relative to the electrically conductive tissue engaging surfaces of the first and second jaw members and is adapted to connect to the electrosurgical energy source. The thermal cutting element is exposed along the length of the electrically conductive tissue engaging surface of the second jaw member and is exposed at the distal end of the jaw housing of the second jaw member.

Provided in accordance with aspects of the present disclosure is an end effector for a surgical instrument that includes a pair of opposing first and second jaw members each including a jaw housing supporting an electrically conductive tissue engaging surface thereon. The electrically conductive tissue engaging surfaces of the first and second jaw members are disposed in opposition relative to one another. The first jaw member is movable relative to the second jaw member to grasp tissue therebetween. The electrically conductive tissue engaging surfaces of the first and second jaw members are adapted to connect to an electrosurgical energy source.

A first thermal cutting element is disposed on the electrically conductive tissue engaging surface of the first jaw member and has a portion that partially extends passed a distal end of the jaw housing of the first jaw member. The thermal cutting element is independently activatable relative to the electrically conductive tissue engaging surfaces and is adapted to connect to the electrosurgical energy source. The thermal cutting element is exposed along the length of the electrically conductive tissue engaging surface of the first jaw member and is exposed at the distal end of the jaw housing of the first jaw member.

An insulative nub is disposed at a distal end of the jaw housing of the second jaw member in vertical registration relative to the first thermal cutting element. The nub and the first thermal cutting element cooperate to pinch tissue upon approximation thereof for tissue treatment.

In aspects according the present disclosure, the nub is made from a thermally insulating material. In other aspects according the present disclosure, the nub is made from an electrically insulating material. In still other aspects according the present disclosure, the nub is made from a thermally insulating and electrically insulating material. In still other aspects according to the present disclosure, the nub is made from an electrically conductive, non-insulative material.

Provided in accordance with aspects of the present disclosure is an end effector for a surgical instrument that includes a pair of opposing first and second jaw members each including a jaw housing supporting an electrically conductive tissue engaging surface thereon. The electrically conductive tissue engaging surfaces of the first and second jaw members are disposed in opposition relative to one another and the first jaw member is movable relative to the second jaw member to grasp tissue therebetween. The electrically conductive tissue engaging surfaces of the first and second jaw members are adapted to connect to an electrosurgical energy source. A first thermal cutting element is operably associated with the first jaw member and a second thermal cutting element is operably associated with the second jaw member. The first thermal cutting element includes a distal tip configured to cover a distal end of the jaw housing of the first jaw member. The second thermal cutting element includes a distal tip configured to cover a distal end of the jaw housing of the second jaw member. The first and second thermal cutting elements are independently activatable relative to the electrically conductive tissue engaging surfaces and are adapted to connect to the electrosurgical energy source. The distal tips of the first and second thermal cutting elements are disposed in vertical registration relative to one another and are configured to cooperate to pinch tissue upon approximation thereof for tissue treatment.

In aspects according the present disclosure, the distal tips of the first and second thermal cutting elements are flush with the distal ends of the jaw housings of the first and second jaw members. In other aspects according the present disclosure, the distal tips of the first and second thermal cutting elements extend relative to the distal ends of the jaw housings of the first and second jaw members.

In aspects according the present disclosure, the distal tips of the first and second thermal cutting elements curve toward one another and cooperate in a beak-like fashion to facilitate pinching tissue therebetween. In other aspects according the present disclosure, the distal tips of the first and second thermal cutting elements curve in the same direction relative to one another to facilitate pinching tissue therebetween.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position;

FIG. 5A is a bottom, perspective view of the first jaw member of the end effector assembly of FIG. 4;

FIG. 5B is a top, perspective view of the second jaw member of the end effector assembly of FIG. 4;

FIGS. 6A-6B are enlarged, front perspective views of a distal end of various embodiments of a thermal cutting element according to the present disclosure;

FIGS. 7A-7D are schematic, side views of various embodiments of the thermal cutting element according to the present disclosure;

FIGS. 8A-8C are various schematic views of additional embodiments of the thermal cutting element according to the present disclosure;

FIGS. 9A-9B are various schematic views of additional embodiments of the thermal cutting element according to the present disclosure;

FIGS. 10A-10B are various schematic views of additional embodiments of the thermal cutting element according to the present disclosure;

FIG. 11 is a schematic, side view of an additional embodiment of the thermal cutting element according to the present disclosure;

FIG. 12A is a schematic view of a representative thermal cutting element according to the present disclosure shown tenting tissue for forming an enterotomy in tissue; and

FIG. 12B is a schematic view of an enterotomy formed within the tissue.

### DETAILED DESCRIPTION

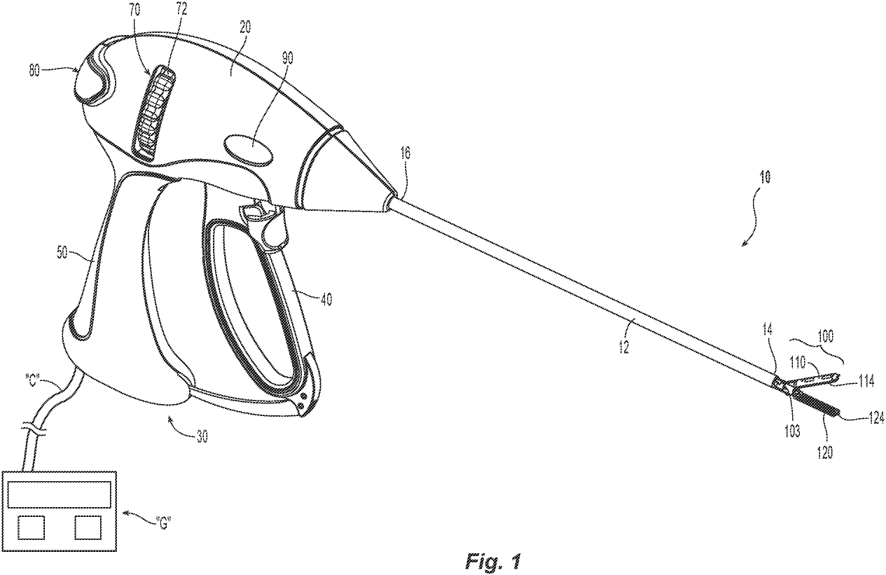

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

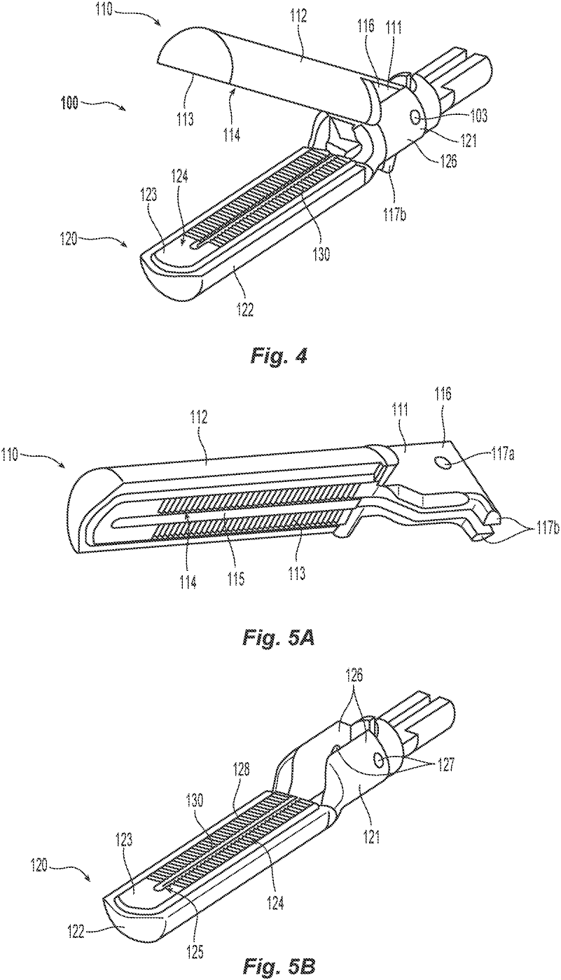

Forceps 10 includes a housing 20, a handle assembly 30, a trigger assembly 60, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto. First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/ desiccating, and/or sealing, tissue. Second activation switch 90 is coupled to thermal cutting element 130 of jaw member 120 (FIG. 4) and the electrosurgical generator “G” for enabling the selective activation of the supply of energy to thermal cutting element 150 for thermally cutting tissue.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

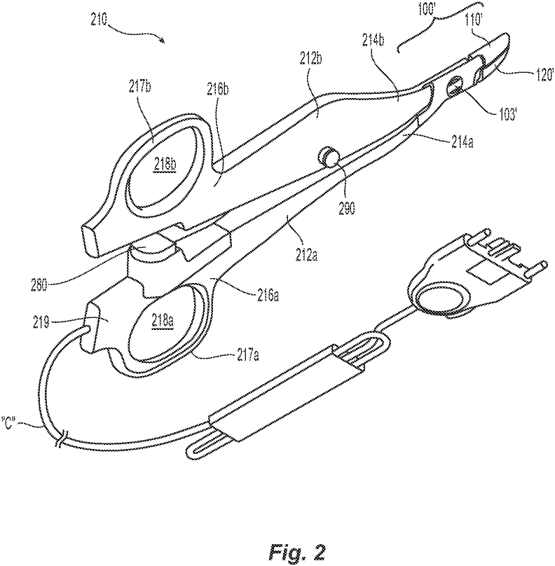

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided for supplying energy to jaw members 110′, 120′ to treat tissue upon sufficient approximation of shaft members 212a, 212b, e.g., upon activation of first activation switch 280 via shaft member 212a. A second activation switch 290 disposed on either or both of shaft members 212a, 212b is coupled to the thermal cutting element (not shown, similar to thermal cutting element 150 of jaw member 120 (FIG. 4)) of one of the jaw members 110′, 120′ of end effector assembly 100′ and to the electrosurgical generator “G” for enabling the selective activation of the supply of energy to the thermal cutting element for thermally cutting tissue.

Jaw members 110′, 120′ define a curved configuration wherein each jaw member is similarly curved laterally off of a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

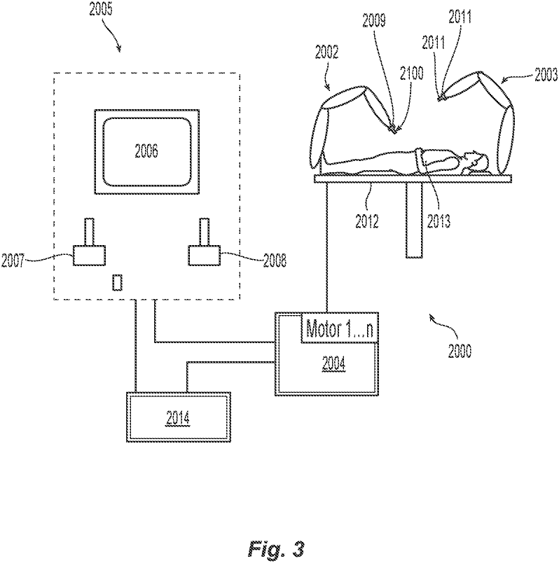

Referring to FIG. 3, a robotic surgical instrument provided in accordance with the present disclosure is shown generally identified by reference numeral 2000. Aspects and features of robotic surgical instrument 2000 not germane to the understanding of the present disclosure are omitted to avoid obscuring the aspects and features of the present disclosure in unnecessary detail.

Robotic surgical instrument 2000 includes a plurality of robot arms 2002, 2003; a control device 2004; and an operating console 2005 coupled with control device 2004. Operating console 2005 may include a display device 2006, which may be set up in particular to display three-dimensional images; and manual input devices 2007, 2008, by means of which a surgeon may be able to telemanipulate robot arms 2002, 2003 in a first operating mode. Robotic surgical instrument 2000 may be configured for use on a patient 2013 lying on a patient table 2012 to be treated in a minimally invasive manner. Robotic surgical instrument 2000 may further include a database 21014, in particular coupled to control device 2004, in which are stored, for example, pre-operative data from patient 2013 and/or anatomical atlases.

Each of the robot arms 2002, 2003 may include a plurality of members, which are connected through joints, and an attaching device 2009, 2011, to which may be attached, for example, an end effector assembly 2100, 2200, respectively. End effector assembly 2100 is similar to end effector assembly 100 (FIG. 4), although other suitable end effector assemblies for coupling to attaching device 2009 are also contemplated. End effector assembly 2200 may be any end effector assembly, e.g., an endoscopic camera, other surgical tool, etc. Robot arms 2002, 2003 and end effector assemblies 2100, 2200 may be driven by electric drives, e.g., motors, that are connected to control device 2004. Control device 2004 (e.g., a computer) may be configured to activate the motors, in particular by means of a computer program, in such a way that robot arms 2002, 2003, their attaching devices 2009, 2011, and end effector assemblies 2100, 2200 execute a desired movement and/or function according to a corresponding input from manual input devices 2007, 2008, respectively. Control device 2004 may also be configured in such a way that it regulates the movement of robot arms 2002, 2003 and/or of the motors.

Turning to FIG. 4, one embodiment of a known end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include the structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114. An outer surface of the jaw housing 112, in such embodiments, may be at least partially coated with an insulative material or may remain exposed. For the purposes herein, the term “insulative” is defined as thermal or electrical conductivity that is lower than the adjacent materials of the jaw members 110, 120. Materials or coatings described herein may be thermally insulative, electrically insulative or both.

In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc.

Referring in particular to FIGS. 4-5B, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 may further include an aperture 117a for receipt of pivot 103 and at least one protrusion 117b extending therefrom that is configured for receipt within an aperture defined within a drive sleeve of the drive assembly (not shown) such that translation of the drive sleeve, e.g., in response to actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc. It is envisioned that the tissue-treating plate, e.g., plate 113 may cover the insulative members 115. The insulative member 115 may be formed from polybenzimidazole or similar materials.

Regardless of the particular configuration of jaw member 110, jaw member 110 may include a longitudinally-extending insulative member 115 extending along at least a portion of the length of tissue-treating surface 114. Insulative member 115 may be transversely centered on tissue-treating surface 114 or may be offset relative thereto. Further, insulative member 115 may be disposed, e.g., deposited, coated, etc., on tissue-treating surface 114, may be positioned within a channel or recess defined within tissue-treating surface 114, or may define any other suitable configuration. Additionally, insulative member 115 may be substantially (within manufacturing, material, and/or use tolerances) coplanar with tissue-treating surface 114, may protrude from tissue-treating surface 114, may be recessed relative to tissue-treating surface 114, or may include different portions that are coplanar, protruding, and/or recessed relative to tissue-treating surface 114. Insulative member 115 may be formed from, for example, ceramic, parylene, nylon, PTFE, or other suitable material(s) (including combinations of insulative and non-insulative materials).

With reference to FIGS. 4 and 5B, as noted above, jaw member 120 includes a structural frame 121, a jaw housing 122, and a tissue-treating plate 123 defining the tissue-treating surface 124 thereof. Jaw member 120 further include a thermal cutting element 130. Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough to pivotably couple jaw members 110, 120 with one another.

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering), etc. Tissue-treating plate 123, as noted above, defines tissue-treating surface 124. A longitudinally-extending slot 125 is defined through tissue-treating plate 123 and is positioned to oppose insulative member 115 of jaw member 110 (FIG. 5A) in the approximated position. Slot 125 may extending through at least a portion of jaw housing 122, a jaw insert (if so provided), and/or other components of jaw member 120 to enable receipt of thermal cutting element 130 at least partially within slot 125. If the thermal cutting element 130 is disposed directly atop the tissue-treating plate 123 (e.g., in the form of a trace), slot 125 may not be necessary. The thermal cutting element 130 may be configured to contact the opposing tissue-treating plate 113 covering the insulative member 115.

Thermal cutting element 130, more specifically, is disposed within longitudinally-extending slot 125 such that thermal cutting element 130 opposes insulative member 115 of jaw member 110 (FIG. 5A) in the approximated position. Thermal cutting element 130 may be configured to contact insulative member 115 (FIG. 5A) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutting element 130 may be surrounded by an insulative member 128 disposed within slot 125 to electrically isolate thermal cutting element from tissue-treating plate 123. Alternatively or additionally, thermal cutting element 130 may include an insulative coating on at least the sides thereof for similar purposes. Thermal cutting element 130 and insulative member 128 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

In embodiments where end effector assembly 100, or a portion thereof, is curved, longitudinally-extending slot 125 and thermal cutting element 130 may similarly be curved, e.g., wherein longitudinally-extending slot 125 and thermal cutting element 130 (or corresponding portions thereof) are relatively configured with reference to an arc (or arcs) of curvature rather than a longitudinal axis. Thus, the terms longitudinal, transverse, and the like as utilized herein are not limited to linear configurations, e.g., along linear axes, but apply equally to curved configurations, e.g., along arcs of curvature. In such curved configurations, insulating member 115 of jaw member 110 (FIG. 5A) is likewise curved.

Generally referring to FIGS. 1-5B, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutting element 130.

Thermal cutting element 130, on the other hand, is configured to connect to electrosurgical generator “G” (FIG. 1) and second activation switch 90 to enable selective activation of the supply of energy to thermal cutting element 130 for heating thermal cutting element 130 to thermally cut tissue disposed between jaw members 110, 120, e.g., to cut the sealed tissue into first and second sealed tissue portions. The thermal cutting element 130 may be controlled by the surgeon, may be automatically controlled by one or more parameters or inputs or feedback associated with the generator “G” or by an algorithm. One or multiple switches 80, 90 may be utilized to accomplish this purpose. For example, if a single switch is utilized, the generator may stop activation of the tissue-treating plates 113, 123 once the seal cycle is complete and automatically initiate activation of the thermal cutting element 130. Other configurations including multi-mode switches, other separate switches, etc. may alternatively be provided. Cross reference is made to U.S. Provisional Pat. Application Serial No. 62/952,232 the entire contents of which being incorporated by reference herein.

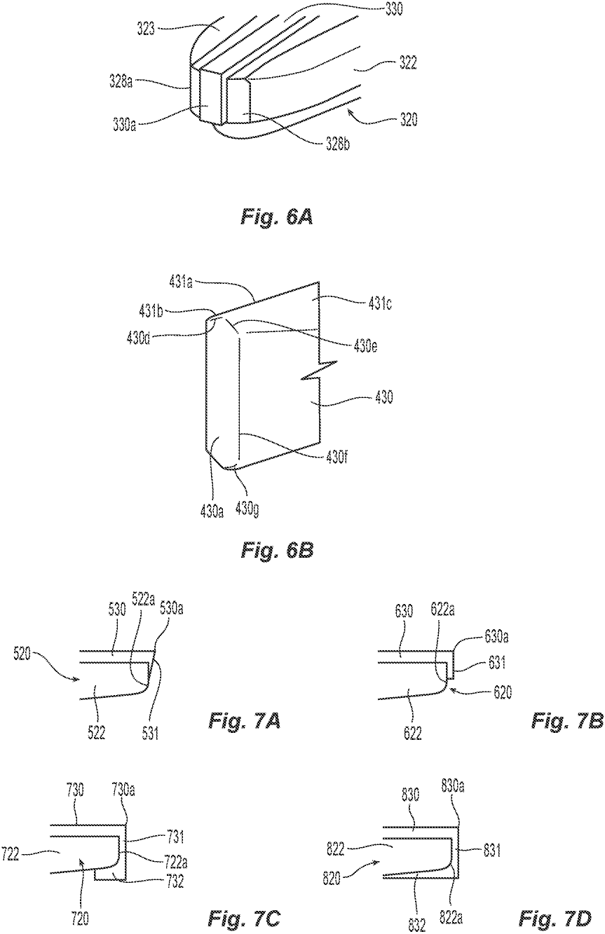

Referring to FIGS. 6A & 6B, another embodiment of the thermal cutting element 330 is shown for use with any of the above-described end effector assemblies and jaw configurations mentioned above. Thermal cutting element 330 is disposed within the jaw housing 322 such that an upper end thereof is exposed relative to the sealing surface 323. Thermal cutting element 330 is disposed between two insulative members 328a, 328b and, in embodiment, is extends relative thereto. Thermal cutting element 330 may also be seated within one or two insulative members 328a, 328b. In this embodiment, the thermal cutting element 330 is configured to be exposed at a distal end 330a thereof to facilitate dissection, back-scoring of tissue and/or facilitate the creation of an enterotomy. Insulative members 328a, 328b are also exposed at respective distal ends thereof to control current or thermal dissipation. Insulators 328a, 328b may also be attached to the thermal cutting element 330 or may be applied as an insulative coating.

FIG. 6B is an alternate embodiment of a thermal cutting element 430 that includes geometry to facilitate cutting and sloughing of tissue post cutting. For example, upper exposed edge of the thermal cutting element 430 includes a cutting spine 431a having a pair of opposing beveled edges 431b, 431c extending away therefrom that are configured to slough tissue away from the cutting spine 431a once cut. In addition, the exposed end 430a of the thermal cutting element 430 is chamfered or rounded to form exposed ends 430d, 430e. Reducing the sharp edges at the exposed end 430a of the thermal cutting element 430 reduces unintended tissue trauma while electrically cutting tissue. Likewise, other exposed edges, e.g., side edges 430f and bottom edges 430g, may be beveled, chamfered or rounded as well depending upon a particular purpose. Configuring the thermal cutting element 430 to be exposed at a distal end 430a thereof facilitates dissection, back-scoring of tissue and/or facilitates the creation of an enterotomy. Insulators 328a, 328b are also exposed at respective ends thereof to control current or thermal dissipation.

It is contemplated that one or both of the jaw members, e.g., jaw member 320, may include geometry to facilitate sloughing of tissue away from the thermal cutting element 330 and/or provide additional tension to the tissue prior to cutting to facilitate tissue separation.

Referring to FIGS. 7A - 7D, several variations of thermal cutting elements are shown. Any of these embodiments of the thermal cutting element may be used with any of the above-described end effector assemblies and jaw configurations mentioned above. FIG. 7A shows one embodiment of a thermal cutting element 530 disposed within the jaw housing 522 of jaw member 520 that includes an exposed distal end 530a configured to extend passed a distal end 522a of the housing 522. In this embodiment, the exposed end 530a of the thermal cutting element 530 includes an inwardly-angled edge 531 configured to improve tissue cutting or dissection at the distal end 530a thereof.

FIG. 7B shows another embodiment of a thermal cutting element 630 disposed within the jaw housing 622 of jaw member 620 that includes an exposed distal end 630a configured to extend passed a distal end 622a of the housing 622. In this embodiment, the exposed end 630a of the thermal cutting element 630 is generally flat to include an exposed flat edge 631 partially along distal end 622a of housing 622. Edge 631 is configured to improve tissue cutting or dissection.

FIG. 7C shows another embodiment of a thermal cutting element 730 disposed within the jaw housing 722 of jaw member 720 that includes an exposed distal end 730a configured to extend passed a distal end 722a of the housing 722. In this embodiment, the exposed end 730a of the thermal cutting element 730 is generally flat to include an exposed flat edge 731 along distal end 722a of housing 722. Distal end 730a includes a trialing edge 732 configured to partially extend back towards the proximal end of the housing 722. The trailing edge 732 (and the exposed flat edge 731) are configured to improve tissue cutting, tissue back-scoring and/or tissue dissection. Trailing edge 732 also enables the user to cut, dissect or score tissue when the end effector, e.g., end effector 100, is pulled proximally. Moreover, tissue may be treated, cut, dissected and/or scored from the opposite or non-treatment (e.g., sealing) side of the jaw housing 722. Distal end 730a may be a rounded edge or other geometry to facilitate cutting and to avoid possible current concentration along an exposed edge.

In embodiment, the beveled (or other geometrically-shaped edge) will facilitate or enhanced back-scoring of tissue as the geometry may produce areas of greater heat concentration to aid in tissue separation, e.g., along a beveled edge.

FIG. 7D shows another embodiment of a thermal cutting element 830 disposed within the jaw housing 822 of jaw member 820 that includes an exposed distal end 830a configured to extend passed a distal end 822a of the housing 822. In this embodiment, the exposed end 830a of the thermal cutting element 830 is generally flat to include an exposed flat edge 831 along distal end 822a of housing 822. Distal end 830a includes a trialing edge 832 configured to extend back towards the proximal end of the housing 822. The trailing edge 832 (and the exposed flat edge 831) are configured to improve tissue cutting, tissue back-scoring and/or tissue dissection. Trailing edge 832 also enables the user to cut, dissect or score tissue when the end effector, e.g., end effector 100, is pulled proximally. Moreover, tissue may be treated, cut, dissected and/or scored from the opposite or non-treatment (e.g., sealing) side of the jaw housing 822.

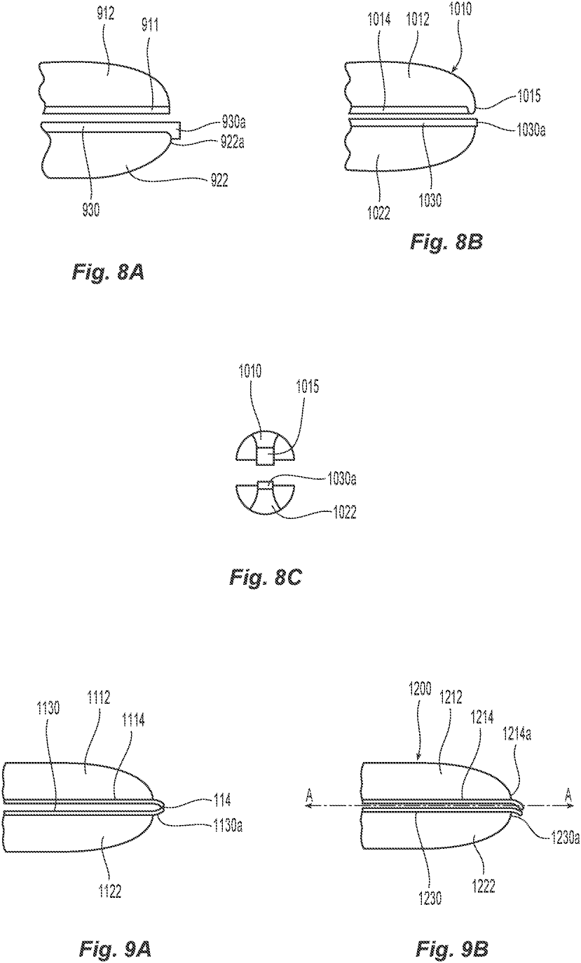

Referring to FIGS. 8A - 8C, other embodiments of thermal cutting elements are shown for use with any of the above-described end effector assemblies and jaw configurations mentioned above. FIG. 8A shows a thermal cutting element 930 disposed within the jaw housing 922 in general opposition to sealing plate 911 of jaw housing 912. Thermal cutting element 930 is exposed at a distal end thereof to include a nub 930a that extends beyond the distal end 922a of the jaw housing 922. Nub 930a facilitates dissection, back-scoring of tissue and/or facilitates the creation of an enterotomy. Insulators 328a, 328b are also exposed at respective ends thereof to control current or thermal dissipation.

FIGS. 8B and 8C show a slight modification of the arrangement shown in FIG. 8A wherein the jaw housing 1012 of jaw member 1010 includes an insulator 1015 at a distal end thereof in vertical registration with sealing plate 1014. Insulator 1015, e.g., plastic, is configured to dissipate thermal or electrical energy at the distal ends of the jaw housings 1012, 1022 during activation of the thermal cutting element 1030.

Referring to FIGS. 9A and 9B, other embodiments of thermal cutting elements are shown for use with any of the above-described end effector assemblies and jaw configurations mentioned above. FIG. 9A shows a first thermal cutting element 1114 disposed within the jaw housing 1112 and a second thermal cutting element 1130 disposed within the jaw housing 1122 in general vertical opposition to thermal cutting element 1114 of jaw housing 1112. Thermal cutting elements 1114 and 1130 include respective distal ends 1114a, 1130a that curve inwardly towards one another in a beak-like manner to allow the user to pinch tissue during manipulation and treatment. Distal ends 1114a, 1130a facilitate dissection, back-scoring of tissue and/or facilitate the creation of an enterotomy.

FIG. 9B shows a first thermal cutting element 1214 disposed within the jaw housing 1212 and a second thermal cutting element 1230 disposed within the jaw housing 1222 in general vertical opposition to thermal cutting element 1214 of jaw housing 1212. Thermal cutting elements 1214 and 1230 include respective distal ends 1214a, 1230a that curve in the same direction relative to a longitudinal axis A-A defined through end effector assembly 1200. This allows the user to pinch tissue during manipulation and treatment. Distal ends 1214a, 1230a facilitate dissection, back-scoring of tissue and/or facilitate the creation of an enterotomy.

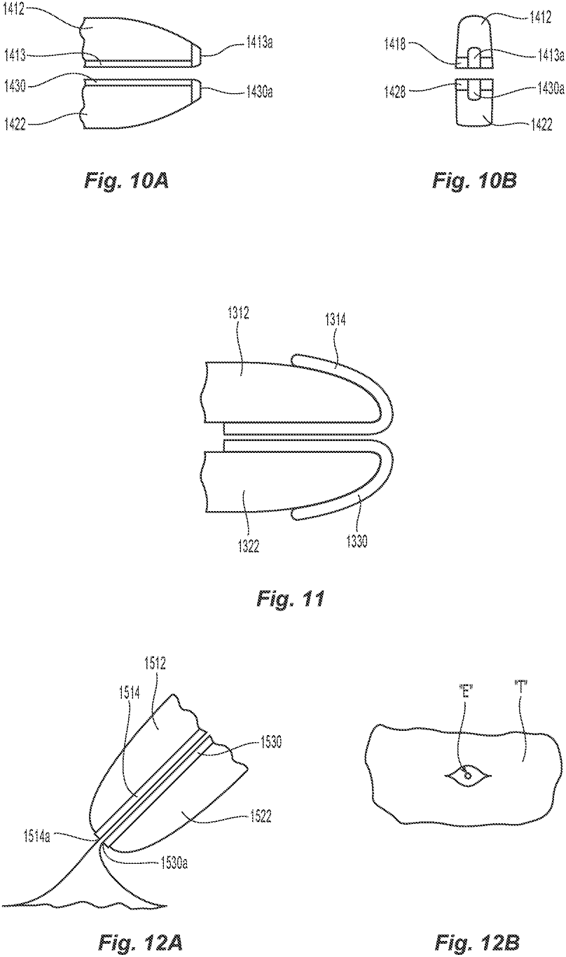

Referring to FIGS. 10A and 10B, another embodiment of a thermal cutting element is shown for use with any of the above-described end effector assemblies and jaw configurations mentioned above. FIG. 10A shows a first thermal cutting element 1413 disposed within the jaw housing 1412 and a second thermal cutting element 1430 disposed within the jaw housing 1422 in general vertical opposition to thermal cutting element 1413 of jaw housing 1412. Thermal cutting elements 1413 and 1430 include exposed respective distal ends 1413a, 1430a that are each housed within an insulator 1418 and 1428 disposed within respective housings 1412, 1422. Distal ends 1413a, 1430a are generally flat and are configured to facilitate dissection, back-scoring of tissue and/or facilitate the creation of an enterotomy. Distal ends 1413a, 1430a may be flush with respective insulators 1418, 1428, extend slightly distally relative thereto, or may be slightly recessed relative thereto depending upon a particular purpose.

Referring to FIG. 11, another embodiment of a thermal cutting element is shown for use with any of the above-described end effector assemblies and jaw configurations mentioned above. FIG. 11 shows a first thermal cutting element 1314 disposed within the jaw housing 1312 and a second thermal cutting element 1330 disposed within the jaw housing 1322 in general vertical opposition to thermal cutting element 1314 of jaw housing 1312. Thermal cutting elements 1314 and 1330 are configured to wrap around the respective jaw housings 1312, 1322 and may be mechanically secured in this fashion. Arranging the thermal cutting elements 1314 and 1330 to wrap around the jaw housings 1312, 1322 may improve tissue cutting, tissue back-scoring and/or tissue dissection. Moreover, this enables the user to cut, dissect or score tissue when the end effector, e.g., end effector 100, is pulled proximally and tissue may be treated, cut, dissected and/or scored from the opposite or non-treatment (e.g., sealing) side of the jaw housings 1312, 1322.

Referring to FIGS. 12A and 12B, one example of creating an enterotomy is shown utilizing any of the aforedescribed thermal cutting elements or jaw configurations mentioned above. For example, FIG. 12A shows first and second opposing thermal cutting elements 1514, 1530 of respective jaw housings 1512, 1522 in an approximated position and pinching tissue “T” at pinch point “A” therebetween at the distal ends 1514a, 1530a thereof. Once tissue is pinched, the tissue “T” is then tented, e.g., pulled. The thermal cutting elements 1514, 1530 are then activated and the tissue “T” disposed between the thermal cutting elements 1514, 1530 at the distal ends 1514a, 1530a thereof is cut leaving an enterotomy “E” in the tissue “T”.

In embodiments, the thermal cutting element of any of the aforedescribed embodiments may be directly attached to one or both of the tissue-contacting surfaces, may be sub-flush or below one or both of the tissue-contacting surfaces and/or may be disposed only on the tip of one or both of the jaw members depending upon a particular purpose or to achieve a particular result.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector assembly for an electrosurgical instrument, comprising:
a pair of opposing jaw members each including a jaw housing supporting an electrically conductive tissue engaging surface thereon, the electrically conductive tissue engaging surfaces disposed in opposition relative to one another, at least one of the pair of jaw members movable relative to the other of the pair of jaw members to grasp tissue therebetween, the electrically conductive tissue engaging surfaces adapted to connect to an electrosurgical energy source; and
a thermal cutting element disposed in at least one of the electrically conductive tissue engaging surfaces, the thermal cutting element independently activatable relative to the electrically conductive tissue engaging surfaces and adapted to connect to the electrosurgical energy source, the thermal cutting element exposed along at least a portion of the length of the at least one electrically conductive tissue engaging surface and including an exposed distal end extending through a distal end of the jaw housing, the exposed distal end of the thermal cutting element configured to facilitate tissue dissection upon activation thereof.

2. The end effector assembly according to claim 1, wherein the thermal cutting element is seated within an insulator disposed in the at least one electrically conductive tissue engaging surface.

3. The end effector assembly according to claim 2, wherein the thermal cutting element is raised relative to the insulator.

4. The end effector assembly according to claim 1, wherein at least a portion of the thermal cutting element includes a beveled surface.

5. The end effector assembly according to claim 1, wherein the thermal cutting element includes a cutting spine disposed along a length thereof having a pair of opposing beveled edges extending away therefrom that are configured to slough tissue away from the cutting spine once the tissue is cut.

6. The end effector assembly according to claim 1, wherein the exposed distal end of the thermal cutting element includes at least one beveled edge.

7. The end effector assembly according to claim 1, wherein the thermal cutting element extends relative to a distal end of the jaw housing.

8. An end effector assembly for an electrosurgical instrument, comprising:
a pair of opposing jaw members each including a jaw housing supporting an electrically conductive tissue engaging surface thereon, the electrically conductive tissue engaging surfaces disposed in opposition relative to one another, at least one of the pair of jaw members movable relative to the other of the pair of jaw members to grasp tissue therebetween, the electrically conductive tissue engaging surfaces adapted to connect to an electrosurgical energy source; and
a thermal cutting element disposed through at least one of the electrically conductive tissue engaging surfaces and having at least a portion thereof disposed through the jaw housing to an opposite end thereof, the thermal cutting element independently activatable relative to the electrically conductive tissue engaging surfaces and adapted to connect to the electrosurgical energy source, the thermal cutting element exposed along the length of the at least one electrically conductive tissue engaging surface and exposed through the jaw housing on the opposite end thereof.

9. The end effector assembly according to claim 8, wherein the thermal cutting element includes an exposed distal end extending through a distal end of the jaw housing, the exposed distal end of the thermal cutting element configured to facilitate tissue dissection upon activation thereof.

10. The end effector assembly according to claim 8, wherein the exposed portion of the thermal cutting element extending through the jaw housing on the opposite end thereof is configured to facilitate back-scoring of tissue along an edge thereof.

11. The end effector assembly according to claim 8, wherein the thermal cutting element is secured within an insulator disposed in the at least one electrically conductive tissue engaging surface.

12. The end effector assembly according to claim 11, wherein the thermal cutting element is raised relative to the insulator.

13. The end effector assembly according to claim 8, wherein at least a portion of the thermal cutting element includes a beveled surface.

14. The end effector assembly according to claim 8, wherein the thermal cutting element includes a cutting spine disposed along a length thereof having a pair of opposing beveled edges extending away therefrom that are configured to slough tissue away from the cutting spine once the tissue is cut.

15. The end effector assembly according to claim 8, wherein the exposed distal end of the thermal cutting element includes at least one beveled edge.

16. The end effector assembly according to claim 8, wherein the thermal cutting element extends relative to a distal end of the jaw housing.
